(19) United States
(12) Patent Application Publication
(10) Pub. No.: US 2023/0337543 A1
(43) Pub. Date: Oct. 19, 2023

(54) FLEXIBLE-PIEZOELECTRIC SENSORS MADE WITH THIN RIBBON CERAMICS AND TRANSITION METAL DICHALCOGENIDES

(71) Applicant: CORNING INCORPORATED, CORNING, NY (US)

(72) Inventors: Ling Cai, Painted Post, NY (US); JaeMyung Chang, Yongin-Si (KR); Yang Liu, Nanjing (CN); Jungwon Seo, Seoul (KR); Cheng-Gang Zhuang, Horseheads, NY (US)

(21) Appl. No.: 18/136,578

(22) Filed: Apr. 19, 2023

Related U.S. Application Data
(60) Provisional application No. 63/332,488, filed on Apr. 19, 2022.

(51) Int. Cl. H10N 30/30; H10N 30/853; H10N 30/87; H10N 30/88; H10N 30/076; A61B 5/00; C04B 41/00; C04B 41/45; C04B 41/50; C04B 41/48; C04B 41/52; C04B 41/89; C04B 41/87; C04B 41/83

(52) U.S. Cl. CPC H10N 30/302; H10N 30/853; H10N 30/87; H10N 30/88; H10N 30/076; A61B 5/6802; C04B 41/009; C04B 41/4531; C04B 41/5054; C04B 41/48; C04B 41/522; C04B 41/89; C04B 41/87; C04B 41/83

(57) ABSTRACT

Embodiments of a sensor are disclosed herein. The sensor includes a ceramic substrate having a first major surface, a second major surface opposite to the first major surface, and a thickness measured from the first major surface to the second major surface. The thickness is from 10 μm to 200 μm. A piezoelectric layer is disposed on the first major surface of the ceramic substrate, and the piezoelectric layer has a thickness of 10 μm or less. At least one electrical contact is disposed on the piezoelectric layer or between the ceramic substrate and the piezoelectric layer or both on the piezoelectric layer and between the ceramic substrate and the piezoelectric layer. A wearable device including such a sensor is also disclosed herein as well as a method of manufacturing same.

### CROSS REFERENCE TO RELATED APPLICATIONS

This application claims the benefit of priority under 35 U.S.C. § 119 to U.S. Provisional Patent Application No. 63/332,488, filed Apr. 19, 2022, the content of which is relied upon and incorporated herein by reference in its entirety.

### BACKGROUND

The disclosure relates generally to piezoelectric sensors and, in particular, to sensors formed from thin piezoelectric films deposited on a ribbon ceramic substrate. Piezoelectric materials produce an electrical response from a mechanical stimulus or a mechanical response from an electrical stimulus. This property of piezoelectric materials can be utilized in sensor devices. However, bulk piezoelectric materials are typically not very sensitive to small stimuli, and therefore, other technologies, such as optical sensors or capacitive sensors, may be utilized in place of piezoelectric sensors for certain applications. Additionally, such piezoelectric materials may be supported on rigid, inflexible substrates, which further diminishes the sensitivity of sensors incorporating piezoelectric materials. These limitations have precluded the use of piezoelectric sensors from certain applications involving a need for conformal coverage of a surface or sufficient sensitivity to detect small perturbances. Notwithstanding, piezoelectric materials and ceramic substrates have high temperature and chemical resistance, and Applicant has determined that it would be desirable to use piezoelectric materials for certain applications.

### SUMMARY

In one aspect, embodiments of the disclosure relate to a sensor. The sensor includes a ceramic substrate having a first major surface, a second major surface opposite to the first major surface, and a thickness measured from the first major surface to the second major surface. The thickness is from 10 μm to 200 μm. A piezoelectric layer is disposed on the first major surface of the ceramic substrate, and the piezoelectric layer has a thickness of 10 μm or less. At least one electrical contact is disposed on the piezoelectric layer or between the ceramic substrate and the piezoelectric layer or both on the piezoelectric layer and between the ceramic substrate and the piezoelectric layer.

In another aspect, embodiments of the disclosure relate to a sensor. The sensor includes a ceramic substrate having a first major surface, a second major surface opposite to the first major surface, and a thickness measured from the first major surface to the second major surface. The thickness is from 80 μm to 150 μm. At least one electrical contact is disposed on at least one of the first major surface or the second major surface. The sensor also includes a first polymer layer and a second polymer layer, and the substrate and at least one electrical contact are disposed between the first polymer layer and the second polymer layer. Further, the ceramic substrate is made of a piezoelectric material.

In still another aspect, embodiments of the disclosure relate to a method of manufacturing a sensor. In the method, a piezoelectric layer made of a transition metal dichalcogenide (TMD) material is sputtered onto a ceramic substrate using an RF magnetron. The ceramic substrate has a first major surface, a second major surface opposite to the first major surface, and a thickness between the first major surface and the second major surface. The thickness is from 10 μm to 200 μm, and the TMD material is disposed on the first major surface of the ceramic substrate. In the method, the TMD material is heat treated at a temperature in a range of 900° C. to 1100° C. for a time of 15 minutes to 45 minutes.

Additional features and advantages will be set forth in the detailed description that follows, and, in part, will be readily apparent to those skilled in the art from the description or recognized by practicing the embodiments as described in the written description and claims hereof, as well as the appended drawings.

It is to be understood that both the foregoing general description and the following detailed description are merely exemplary, and are intended to provide an overview or framework to understand the nature and character of the claims.

### BRIEF DESCRIPTION OF THE DRAWINGS

The accompanying drawings are included to provide a further understanding and are incorporated in and constitute a part of this specification. The drawings illustrate one or more embodiment(s), and together with the description serve to explain principles and the operation of the various embodiments.

FIG. 1 depicts a schematic, cross-sectional view of a sensor having a transition metal dichalcogenide (TMD) layer piezoelectric material deposited on a flexible ribbon ceramic, according to an exemplary embodiment;

FIG. 2 depicts an example of a ceramic ribbon substrate having a piezoelectric layer and electrical contacts deposited thereon, according to an exemplary embodiment;

FIG. 3 depicts a ribbon ceramic being bent to demonstrate the flexibility of the substrate of the sensor; according to an exemplary embodiment;

FIG. 4 is a graph of the mechanical response (displacement) as a function of applied voltage to various piezoelectric materials, including two comparative examples and two examples according to exemplary embodiments;

FIG. 5 is a schematic representation of a roll-to-roll processing line for preparing a ribbon ceramic substrate, according to an exemplary embodiment;

FIG. 6 is a schematic representation of an RF magnetron sputtering chamber for applying the piezoelectric layer, according to an exemplary embodiment;

FIGS. 7A and 7B depict MoS₂ monolayers deposited on alumina and zirconia ribbon substrate, respectively, after heat treating, according to exemplary embodiments;

FIG. 8 depicts a sensor having a piezoelectric layer and a flexible ceramic ribbon substrate used in a pulse sensor, according to an exemplary embodiment;

FIG. 9 is a schematic perspective view of a sensor, according to an exemplary embodiment;

FIG. 10 is a wearable device incorporating a sensor constructed as shown in FIG. 9, according to an exemplary embodiment;

FIG. 11 depicts a graph of the piezoelectric charge constant measured at eight points on the piezoelectric layer of the wearable device shown in FIG. 10, according to an exemplary embodiment; and

FIGS. 12 and 13 depict various piezoelectric responses to the bending and unbending of the wearable device of FIG. 10 over the course of various cycles, according to exemplary embodiments.

### DETAILED DESCRIPTION

Referring generally to the figures, various embodiments of a piezoelectric sensor are disclosed. As will be discussed more fully below, the piezoelectric sensor is formed from a flexible ceramic substrate having a thin piezoelectric layer deposited thereon. In particular embodiments, the piezoelectric layer is comprised of transition metal dichalcogenide (TMD), which is, in particular, deposited in a monolayer on a ribbon ceramic using RF magnetron sputtering. In other particular embodiments, the piezoelectric layer is comprised of lead zirconate titanate (PZT) or lead magnesium niobate-lead titanate (PMN-PT), which can be prepared, e.g., using a sol-gel process to provide thin layers, such as down to about 2 μm. In still other particular embodiments, the piezoelectric sensor includes a substrate formed from a ribbon ceramic piezoelectric material such that no additional layer is needed to provide the piezoelectric sensing. Advantageously, the thinness of the piezoelectric layer and substrate enhance the sensitivity of the sensor device to mechanical stimuli. Further, the sensor can be conformal to various surfaces, including to skin. In this regard, the increased sensitivity and conformality allow the piezoelectric sensor to be utilized as a wearable sensor for monitoring of health-related information, such as heart rate. These and other aspects and advantages of the disclosed flexible piezoelectric sensor will be discussed in relation to the exemplary embodiments provided below and in the figures. The discussion is provided by way of illustration and not by way of limitation.

FIG. 1 depicts an embodiment of a sensor 100. The sensor 100 includes a substrate 102 having a first major surface 104 and a second major surface 106. The second major surface 106 is opposite to the first major surface 104, and a thickness T is defined between the first major surface 104 and the second major surface 106. A piezoelectric layer 108 is deposited on at least a portion of the first major surface 104. The piezoelectric layer 108 is deposited in such a way as to exhibit a piezoelectric effect, in particular by depositing the piezoelectric layer 108 with a thin thickness as will be discussed below. For example, in one or more embodiments, the piezoelectric layer 108 is a transition metal dichalcogenide (TMD) material, which exhibits piezoelectricity when deposited in small thicknesses, such as in a monolayer. In one or more other embodiments, the piezoelectric layer 108 is comprised of a PZT (Pb(ZrₓTi₁₋ₓ)O₃) or PMN-PT (Pb(MgₓNb₁₋ₓ)O₃—PbTiO₃). The sensor 100 also includes electrical contacts 110. In the schematic cross-sectional view depicted in FIG. 1, one electrical contact 110 can be seen between the first major surface 104 of the substrate 102 and the piezoelectric layer 108, and another electrical contact 110 can be seen deposited on the piezoelectric layer 108. In the sensor 100, the electrical contacts 110 allow for sensing of the electrical response that the piezoelectric layer 108 exhibits when undergoing a mechanical stress.

In one or more embodiments, substrate 102 is a ceramic material. In one or more such embodiments, the substrate 102 is formed from a ribbon ceramic. A ribbon ceramic is a ribbon formed from a ceramic material. As used herein, a “ribbon” is a strip of material in which its length is much greater than its width and its width is much greater than its thickness. In one or more embodiments, a ribbon ceramic may have a length of at least 0.5 m and up to 100 m. Further, in one or more embodiments, a ribbon ceramic may have a width of at least 10 mm and up to 100 mm or even up to 200 mm. In one or more embodiments, the thickness of the ribbon ceramic (i.e., distance between the first major surface 104 and the second major surface 106) is in a range from 10 μmm to 200 μmm, in particular in a range from 20 μmm to 120 μm, and most particularly in a range from 20 μm to 80 μm. While the substrate 102 of the sensor 100 may be formed from a ribbon ceramic, including through a process as described below, the final sensor 100 product may be singulated from the ribbon ceramic to a smaller length and width than the described ribbon material.

In one or more embodiments, the substrate 102 is formed from silica (SiO₂), zirconia (ZrO₂), stabilized zirconia (e.g., yttria- or scandia-stabilized zirconia), or alumina (Al₂O₃). In one or more other embodiments, the substrate 102 is formed from a piezoelectric material, such as PZT or PMN-PT, and the sensor 100 may not include a separate piezoelectric layer 108. Advantageously, a substrate 102 made of one of these materials in the ribbon form has a thickness and flexibility that allows the substrate 102 to flex with the piezoelectric layer 108 or flex to exhibit a piezoelectric effect in a manner that enhances the sensitivity of the sensor 100 when experiencing a mechanical stimulus. That is, by providing for increased flexing of the substrate 102, the electrical response of the piezoelectric layer 108 or of the piezoelectric substrate 102 can be increased for a given mechanical stimulus. Additionally, the ribbon ceramic of the substrate 102 has a small grain size that helps to increase flexibility, thereby enhancing the piezoelectric effect of the sensor 100.

In one or more embodiments, the piezoelectric layer 108 is formed from such TMD materials as molybdenum disulfide (MoS₂), tungsten disulfide (WS₂), tungsten diselenide (WSe₂), molybdenum ditelluride (MoTe₂), or tungsten ditelluride (WTe₂). In one or more embodiments, the piezoelectric layer 108 is formed as a monolayer on the substrate 102. The monolayer TMD materials are not structurally centrosymmetric and exhibit a piezoelectric effect. Advantageously, as compared to conventional bulk piezoelectric crystals, deflection of a monolayer material provides in-plane mechanical stimulation sufficient to produce an electric response without high mechanical loads, thereby increasing the sensitivity of the sensor 100 in which the monolayer material is incorporated. While in one or more embodiments a monolayer TMD is preferred, the TMD may be provided in multiple layers, in particular in an odd number of layers, such as up to eleven layers. Further, the TMD materials are synthesized at process temperatures in the range of about 700° C. to about 1000° C., which means that, once deposited, the TMD materials will be relatively chemically stable and electrically isolated.

In one or more embodiments, the substrate 102 is carried on a support 112. In one or more embodiments, the support 112 allows for flexing of the substrate 102. That is, the support 112 defines an interior cavity 114 into which the substrate 102 can flex when experiencing a mechanical stimulus. As mentioned above, the thinness and small grain size of the substrate 102 provides flexibility for the substrate to flex into the interior cavity 114. The support 112 can be made of any of a variety of materials that can be manufactured to have an interior cavity 114 and be attached to the substrate 102. In one or more embodiments, the support 112 is comprised of silicon, silica (SiO₂), zirconia (ZrO₂), stabilized zirconia (e.g., yttria- or scandia-stabilized zirconia), alumina (Al₂O₃), or a piezoelectric material (such as PZT or PMN-PT). In one or more embodiments, the support 112 is attached to the substrate 102 by, e.g., organic adhesives, metal-metal diffusion bonding, and soldering, among other processes.

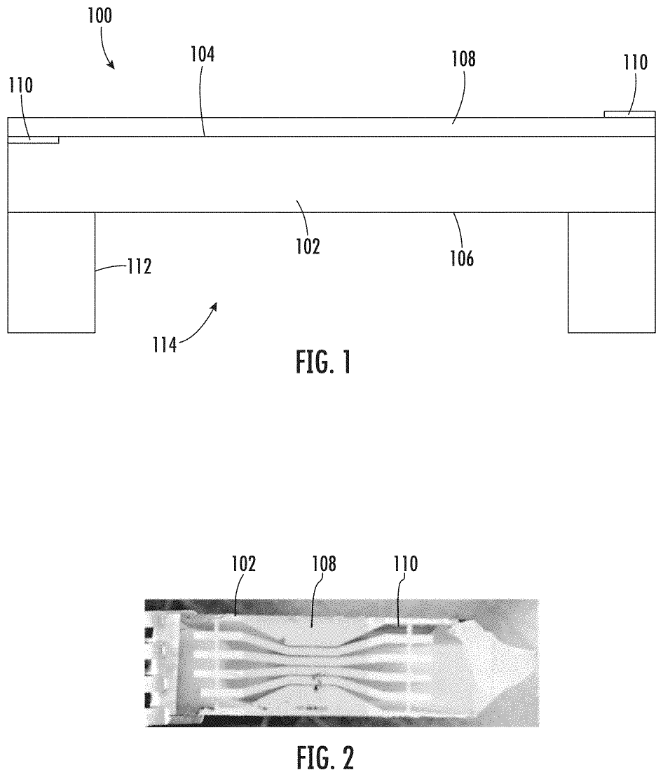

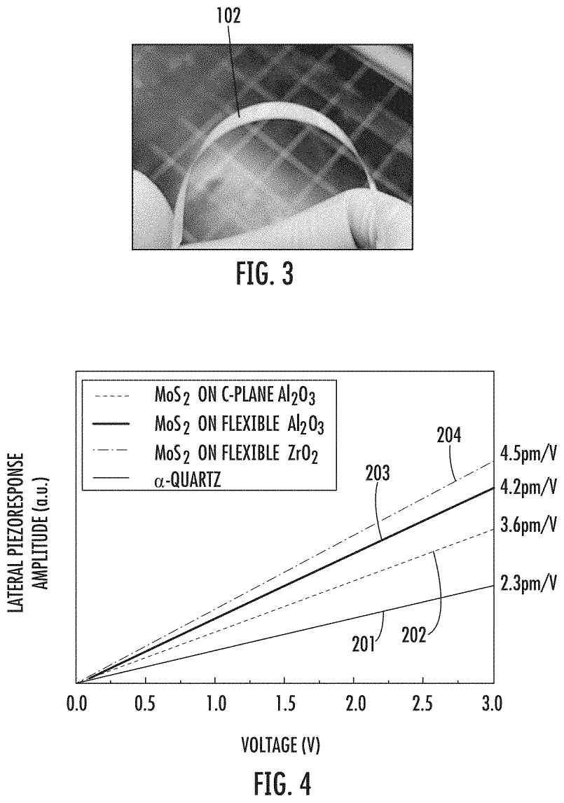

FIG. 2 depicts an example of a substrate 102 having electrical contacts 110 and a piezoelectric layer 108 deposited thereon. FIG. 3 depicts the substrate 102 of FIG. 2 being flexed into a curved shape to demonstrate the flexibility of the substrate 102. As mentioned above, the increased flexibility of the substrate 102 enhances the sensitivity of the sensor 100 because a mechanical stimulus will produce an increased piezoelectric effect.

FIG. 4 provides a graph of the mechanical response as a function of applied voltage for two substrates 102 and respective piezoelectric layers 108 according to the present disclosure and for two comparative piezoelectric structures. The electrical response to an applied mechanical stimulus is proportional to the mechanical response to an applied electrical stimulus as shown in FIG. 4. In the first comparative case 201, a bulk α-quartz crystal was subjected to an applied voltage, and it was found that the bulk a-quartz crystal deflected about 2.3 pm/V. In the second comparative case 202, MoS₂ was deposited on c-plane alumina, and it was found that the substrate and piezoelectric layer deflected about 3.6 pm/V. In the first example embodiment case 203, the substrate 102 was flexible ribbon alumina with an MoS₂ piezoelectric layer 108, and it was found that the substrate 102 deflected 4.2 pm/V. In a second example embodiment case 204, the substrate 102 was flexible zirconia with an MoS₂ piezoelectric layer 108, and it was found that the substrate 102 deflected 4.5 pm/V. Thus, the combination of flexible substrate 102 and piezoelectric layer 108, in particular monolayer TMD, provides greater sensitivity to stimuli.

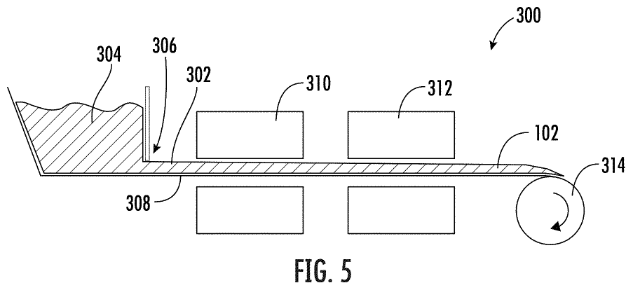

FIG. 5 depicts a roll-to-roll processing line 300 for the manufacture of a ribbon substrate according to an example embodiment. In one or more embodiments, the process begins with the formation of a green tape 302 from a slurry or paste precursor 304. In one or more embodiments, the precursor 304 includes a powder component, a binder, and a solvent. The powder component includes a powdered form of the ribbon substrate, e.g., a silica, a zirconia, an alumina, a PZT, or a PMN-PT powder. The binder holds the powder component together in the form of the green tape prior to sintering. Suitable binders include, e.g., polyvinyl butyral (PVB), acrylic polymers, or polyvinyl alcohol, among others. The powder component and binder are dispersed in the solvent, which allows the precursor 304 to be extruded or formed into the ribbon structure. In embodiments, the precursor may contain other additives that aid in processing, such as 0.1% to 5% by weight of a dispersant, plasticizer, or antioxidant, among other possibilities.

As shown in FIG. 5, the precursor 304 is extruded or tape cast through die 306 into a green tape 302 having the desired thickness and carried on a conveyor or carrier tape 308. In one or more embodiments, the thickness may be in the range of from 10 μm to 200 μm, in particular 20 μm to 150 μm. For a substrate 102 that is a piezoelectric material, the thickness may be in the range of about 80 μm to 150 μm, in particular 100 μm to 150 μm. In embodiments, the green tape 302 is dried at drying station 310 to remove a substantial portion of the solvent, leaving primarily the powder component and binder. In embodiments, drying may occur at ambient temperature or at a slightly elevated temperature of, e.g., 60° C. to 80° C. (or begin at an ambient temperature and transition to an elevated temperature). Additionally, in embodiments, air is circulated to enhance drying. Upon drying, the green tape 302 is debound and sintered in heating chamber 312. In embodiments, the green tape 302 is heated for a time and to a temperature (e.g., 175° C. to 350° C.) at which the polymer binder and any other organics are burned off, and then the green tape 302 is heated for a time and to a temperature (e.g., 500° C. to 1350° C.) at which the powder is sintered. In one or more embodiments, the ribbon substrate 102 has a porosity in a range of 0.1% to 30% after sintering and/or has on average a grain size of from 10 nm to 50 μm. In embodiments, as shown, the sintered, ribbon substrate can be rolled on a spool 314.

Upon formation of the ribbon ceramic substrate, a set of electrical contacts 110 may be formed on the substrate 102. In one or more embodiments, the electrical contacts 110 can be formed through a variety of wet deposition processes, such as dip coating, slot die coating, doctor blading, spin coating, or electrodeposition, among others. In one or more embodiments, the electrical contacts 110 can be formed through a variety of dry deposition processes, such as sputtering, physical vapor deposition, or chemical vapor deposition, among others. In one or more embodiments, deposition of the electrical contact 110 material may be followed by etching, lithography, or other ways of selective removal of contact material to provide, e.g., a plurality of leads. In one or more embodiments, the electrical contacts 110 are formed from gold, silver, platinum, rhodium, copper, aluminum, or titanium, among others.

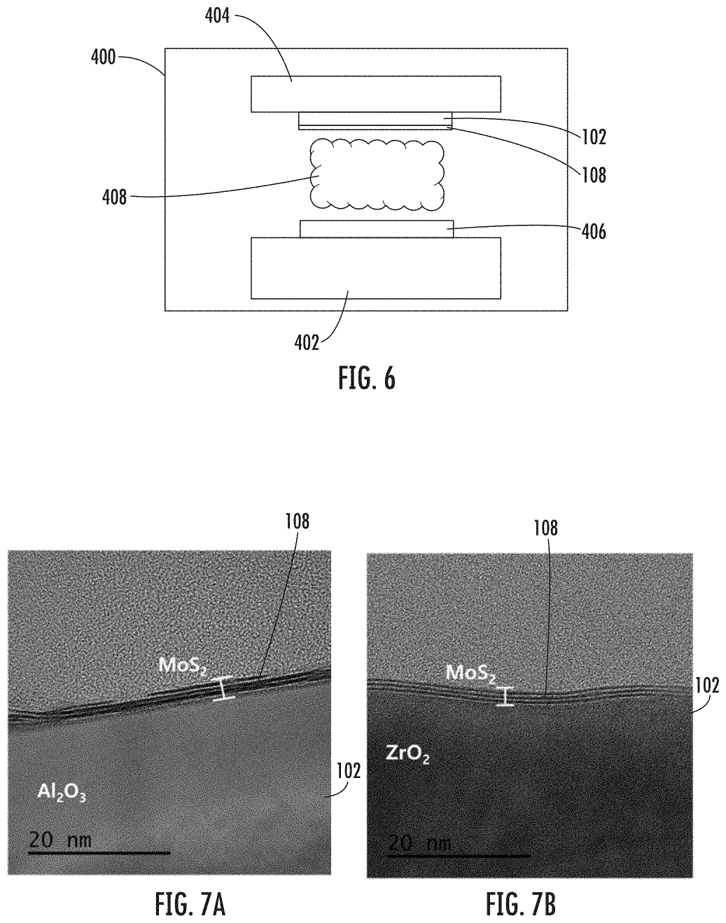

After formation of the set of electrical contacts 110, in one or more embodiments, the piezoelectric layer 108 is formed from a TMD material via RF magnetron sputtering. As shown in FIG. 6, RF magnetron sputtering involves positioning the substrate 102 in a chamber 400. The chamber 400 has a cathode 402 and an anode 404, and the substrate 102 is positioned between the cathode 402 and anode 404, in particular near the anode 404. A target 406 of the TMD material is positioned near to the cathode 402. For example, to deposit an MoS₂ piezoelectric layer 108 on the substrate 102, the target 406 may be a vanadium-doped molybdenum disulfide (V-MoS₂) target. Argon gas is pumped into the chamber 400. Further, in one or more embodiments, the argon gas may be mixed with a gas that facilitates formation of the piezoelectric layer 108. For example, hydrogen sulfide (H₂S) gas may be pumped into the chamber 400 to form a MoS₂ piezoelectric layer 108. During sputtering, an RF electric field is formed between the cathode 402 and anode 404, which ionizes the argon gas to form a plasma 408. The argon ions contact the target 406 and sputter the TMD material, and atoms of the TMD material are drawn toward the anode 404 and deposited on the substrate 102. One advantage of RF magnetron sputtering is that the number of piezoelectric layers applied to the substrate 102 can be closely controlled based on the amount of time that the RF magnetron sputtering is performed. In one or more embodiments, RF magnetron sputtering is carried out under the following conditions: temperature of from 500° C. to 700° C., working pressure of 1 Pa, RF power of 30 W, and distance between the target 406 and the substrate 102 of about 12 cm.

After RF magnetron sputtering, the deposited piezoelectric layer 108 is heat treated to improve crystallinity. In one or more embodiments, the piezoelectric layer 108 is heat treated at a temperature in a range of 900° C. to 1100° C., in particular in a range of 990° C. to 1010° C. In one or more embodiments, the piezoelectric layer 108 is heat treated for 15 minutes to 45 minutes, in particular 25 minutes to 35 minutes. Further, in one or more embodiments, the heat treating takes place in an atmosphere of a flowing chalcogenide gas, such as a hydrogen chalcogenide gas (e.g., hydrogen sulfide or hydrogen selenide). In a particular embodiment in which an MoS₂ piezoelectric layer 108 was formed, the piezoelectric layer 108 was heat treated at 1000° C. for 30 minutes in an atmosphere of flowing H₂S gas. FIGS. 7A and 7B depict SEM images of an MoS₂ piezoelectric layer 108 applied to an alumina ribbon substrate 102 (FIG. 7A) and to a zirconia ribbon substrate 102 (FIG. 7B). In particular, the images show three distinct layers of MoS₂ deposited onto the respective substrates. In general, the grain size of the zirconia substrate 102 tends to be smaller than the grain size of the alumina substrate 102. As such, the zirconia substrate 102 also tends to be more flexible than the alumina substrate 102.

After the piezoelectric layer 108 is heat treated, another set of electrical contacts 110 is formed over the piezoelectric layer 108. The electrical contacts 110 can be formed in the same manner as described above in relation to the electrical contacts formed between the substrate 102 and the piezoelectric layer 108. While the foregoing discussion describes electrical contacts 110 formed on both sides of the piezoelectric layer 108, the electrical contacts 110 may be applied to just one side of the piezoelectric layer 108 in one or mor other embodiments.

After preparing the substrate 102 by depositing the electrical contacts 110 and the piezoelectric layer 108, the substrate 102 is attached to the support 112. As mentioned above, the support 112 may be attached to the substrate 102 by, e.g., organic adhesives, metal-metal diffusion bonding, and soldering, among other processes.

For a TMD piezoelectric layer 108, a monolayer of TMD material is preferably applied to the substrate 102 to provide a sensor 100 having enhanced sensitivity to mechanical stimuli. Such a sensor 100 provides several advantages and can be used in a variety of contexts. Advantages of such a sensor 100 include temperature and chemical stability, enhanced sensitivity, and scalability. In particular, the piezoelectric layer 108 of the sensor is grown on the substrate 102 through RF magnetron sputtering according to the present disclosure instead of the conventional process of transferring of TMD flakes onto the substrate. TMD flakes have been deposited through chemical vapor deposition onto various substrates, such as silica/silicon, sapphire, polymers, and metal foils, but the flakes overlap, creating non-uniform strains in and among the TMD flakes. Further, TMD flakes are more loosely bonded to the substrate and tend to fall off of the substrate more easily than the TMD piezoelectric layer 108.

Utilizing the ribbon ceramic as the substrate 102 improves the sensitivity of the sensor to mechanical stimuli. As discussed above, the ribbon ceramic is processed in the desired thickness for use as the substrate 102 using a roll-to-roll manufacturing process. Conventional substrates are typically sintered in much larger thicknesses and then lapped and polished to the desired thickness, which is a much more time-consuming and costly process. Additionally, such lapped and polished substrates do not possess the high elastic modulus and low grain size of the ribbon ceramic substrates used in the sensors 100 according to the present disclosure. Still another advantage of the presently disclosed substrate 102 with piezoelectric layer 108 is the ability to scale the sensors for large area applications, including microfluidics, acoustic sensors, and pressure monitoring.

One particular application for which use of the disclosed sensor 100 is envisioned is high temperature, non-contact pressure sensors. Because of the flexibility of ribbon ceramics utilized as the substrate 102, the sensor 100 can be utilized to monitor ambient pressure inside turbine engines, including air flow at an intake or an out exhaust. In contrast to conventional piezoelectric sensors, seismic mass is not required so that footprint of the disclosed sensors 100 may be significantly reduced.

Another application for which use of the disclosed sensor is envisioned is acoustic sensors for consumer electronics. Micro-electromechanical systems (MEMS) based on capacitive acoustic sensors have been widely employed in microphones for consumer electronics such as smart phones, voice assistants, and ear buds. However, piezoelectric sensors exhibit better performance in terms of resolution and sensitivity and have simpler structures than capacitive sensors. In view of the thinness and sensitivity, the disclosed sensor 100 having a flexible substrate 102 and piezoelectric layer 108 is envisioned to be a replacement for capacitive sensors in such consumer electronics as smart phones, voice assistants, and ear buds.

Still another application for which use of the disclosed sensor is envisioned is flexible and conformable contact pressure sensors. As described above, the thin piezoelectric layer 108 on a substrate 102 formed from a ribbon ceramic can be used for contact pressure sensors, such as a haptic sensor, with good piezoelectric performance. In comparison to conformal sensors that use a polymer substrate or insulated metal foil, the ribbon ceramic substrate 102 of the disclosed sensor 100 has greater mechanical integrity and a higher elastic modulus. Further, the direct formation of monolayer TMD on the substrate 102 allows for creation of large area sensors with conformal coverage.

FIG. 8 depicts an embodiment of a flexible and conformable contact pressure sensor 500 in the form of a pulse sensor on a wrist 501 of a wearer. As can be seen in FIG. 8, the sensor 500 is flexible enough to conform to the shape of the wearer's wrist 501, which allows for the piezoelectric layer to pick up the pulsations of blood flowing through, e.g., the radial artery. While a pulse sensor on the wrist 501 is depicted, the sensor 500 could instead be positioned on a wearer's neck or chin for sleep analysis, chest for breath analysis, abdomen for gastrological monitoring, or arms or legs for muscle movement, among other possibilities.

In the embodiment depicted, the mechanical stimulus of the pulse causes an electrical reaction of the piezoelectric layer that can be conducted through the electrical contacts. In this way, the electrical response of the piezoelectric layer can be correlated to the wearer's pulse. Advantageously, the sensor 500 is self-powered in that the mechanical stimulus causes generation of the electrical impulse. Certain conventional pulse monitors use optical sensors that require a separate power source. Thus, when incorporated into a wearable device, such as a fitness watch, the sensor 500 does not consume as much energy as the conventional optical sensors used to measure a wearer's pulse. The energy savings are even greater when additional functions, such as monitoring of the wearer's electrocardiogram (ECG), sleep cycle, breathing, neurological health, and running pace, are considered. That is, battery life of the wearable device can be extended by not diverting energy to the sensor 500 and instead using mechanical power of the piezoelectric sensor 500 to monitor these health aspects. Additionally, the sensor 500 is essentially a 2D sensor for which sensitivity can be improved by increasing the 2D coverage area. Optical sensors that are conventionally used for health monitoring in wearable devices are limited in their sensitivity based on the optical spot size.

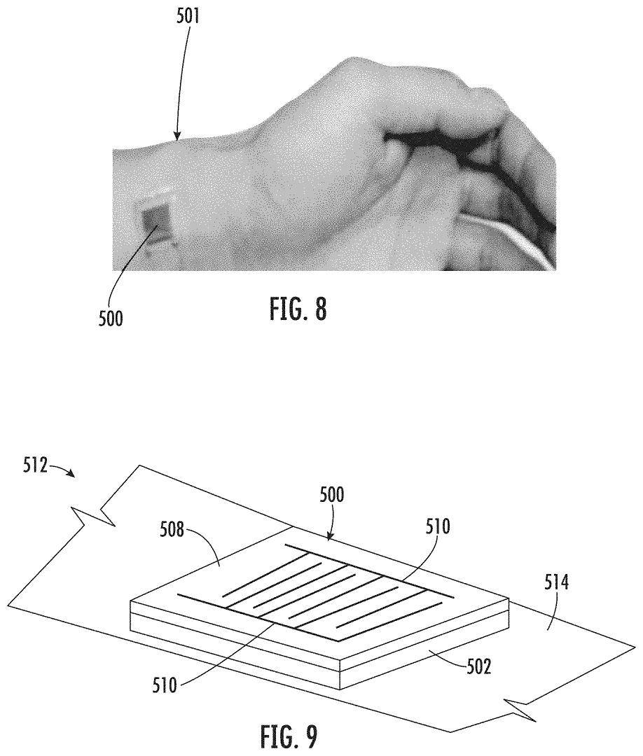

FIG. 9 depicts a schematic perspective view of a sensor 500 including a flexible ribbon ceramic substrate 502, a piezoelectric layer 508, and electrical contacts 510, which are depicted as interdigitated contacts. As discussed above, the piezoelectric layer 508 may be made from a TMD material (particularly 11 or less atomic layers) or from a thin film of PZT or PMN-PT (e.g., having a thickness in a range from 1 μm to 10 μm, in particular 2 μm to 5 μm). The sensor 500 can be integrated into a wearable device or a patch 512. In particular, FIG. 9 depicts the sensor 500 laminated to a polymer layer 514. For example, the polymer layer 514 may integrate the sensor 500 into a wearable band, such as a watch band. Further, while the sensor 500 is depicted as laminated to one polymer layer 514, the sensor 500 in one or more other embodiments may be laminated between two polymer layers 514. In one or more embodiments, the polymer layer or layers 514 may be made of polyethylene terephthalate (PET).

In one or more embodiments, the substrate 502 for the sensor 500 integrated into the wearable device 512 is selected to be a zirconia substrate because it is more flexible than other ribbon ceramic substrates. In one or more particular embodiments, the substrate is stabilized zirconia, such as yttria stabilized zirconia (e.g., zirconia stabilized with 3 mol % yttria). Ribbon substrates of zirconia can be attached conformally on human skin, and as discussed above, the zirconia ribbon substrate has good surface flatness to support the growth of a high quality piezoelectric layer. Additionally, being a ribbon ceramic material, the zirconia substrate exhibits a relatively high flexural strength compared to other lapped and polished thin ceramic materials, such as mica.

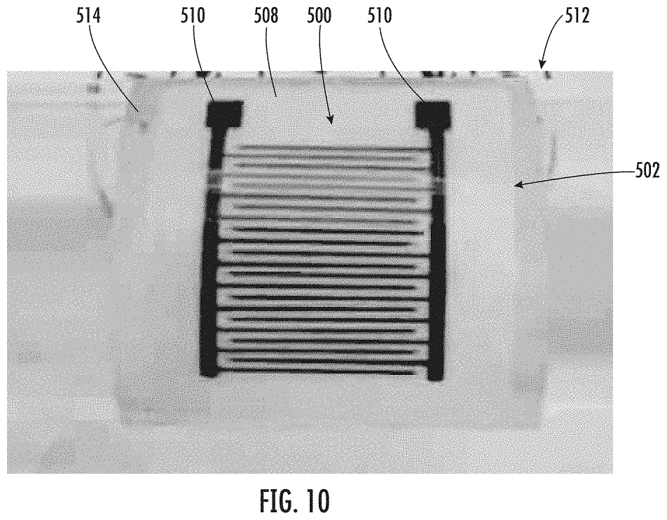

FIG. 10 depicts an example embodiment of a wearable device 512 prepared as described in relation to FIG. 9. The substrate 502 was zirconia stabilized with 3 mol % yttria (3YSZ). A piezoelectric layer 508 made of lead zirconate titanate (PZT) having the formula Pb[Zr_{0.52}Ti_{0.48}]O₃ was grown on the substrate 502 using a sol-gel process. In the sol-gel process, PZT was grown from a solution comprising 2-methoxyethanol as a solvent and acetylacetone as a chelating agent. The PZT solution was applied to the substrate, and the layers were annealed at about 700° C. Interdigitated electrical contacts 510 made of titanium (20 nm) or platinum (100 nm) were formed on the piezoelectric layer 508. Further, the sensor 500 was laminated between PET polymer layers 514 to form the wearable device 512.

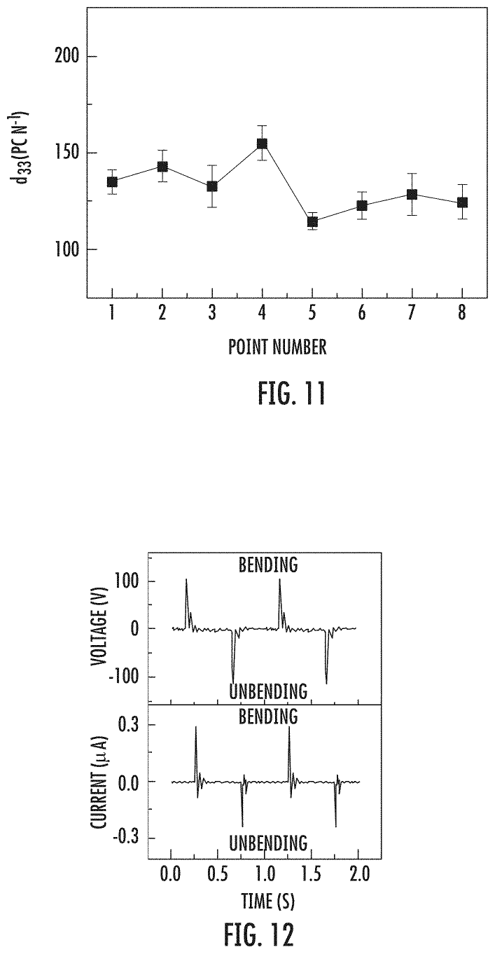

Using the wearable device 512 shown in FIG. 10, the piezoelectric charge constant d₃₃ was measured on the PZT piezoelectric layer 508. Because of the grain structure of 3YSZ substrate 502, the PZT piezoelectric layer 508 is weakly poled in the as-grown state. Nevertheless, the measured piezoelectric charge constant d₃₃ of the as-grown PZT piezoelectric layer 508 averaged greater than 100 pC/N when measured at eight points across the PZT piezoelectric layer 508 as shown in FIG. 11. The piezoelectric charge constant d₃₃ can be further increased after poling with moderate electric field at a temperature of about 100° C. Additionally, it is believed that utilizing a piezoelectric substrate 502 and not separate ribbon ceramic substrate 502 and piezoelectric layer 508 would increase the piezoelectric charge constant d₃₃. In particular, the inventors surmise that the piezoelectric charge constant d₃₃ for a piezoelectric substrate having a thickness of 100 μm to 150 μm would be around 400 pC/N to 500 pC/N. In part, the higher piezoelectric charge constant d₃₃ is believed to result from the higher sintering temperature to which the piezoelectric substrate 502 can be subjected.

In one or more embodiments in which the piezoelectric sensor 500 includes a piezoelectric substrate 502 without a separate piezoelectric layer 508, the piezoelectric substrate 502 has electrodes 510 deposited directly thereon. Further, in one or more such embodiments, the piezoelectric substrate 502 and electrodes 510 are disposed between polymer layers 514. In such embodiments, the polymer layers 514 protect the electrodes 510 from being worn away from the substrate 502.

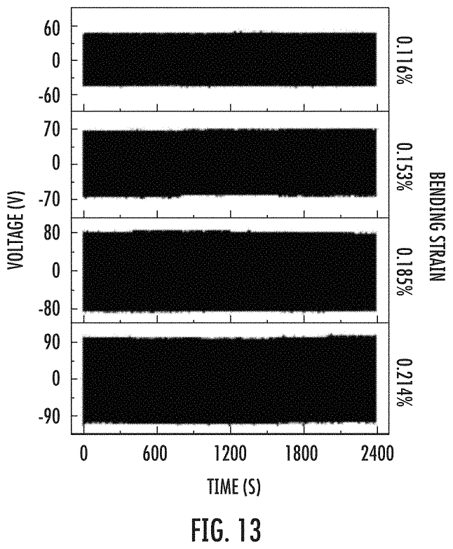

FIGS. 12 and 13 depict the bending and unbending characteristics of the wearable device 512 incorporating the sensor 500. Referring first to FIG. 12, the voltage response to bending and unbending is shown in the upper portion of the graph. As can be seen, the sensor 500 provides consistent voltage outputs of +/−100 V for bending and unbending. The lower portion of the graph in FIG. 12 depicts the current (μA) produced during bending and unbending. As can be seen, bending produces a current output of about 0.3 μA, and unbending produces a slightly lower magnitude of current output at about −0.25 μA.

FIG. 13 depicts just the voltage response to bending and unbending. While FIG. 12 depicted two cycles of bending/unbending, FIG. 13 depicts 2400 cycles for various different bending strains (0.116%, 0.153%, 0.185%, and 0.214%). As can be seen, the increasing bending strain produces an increasing voltage response. However, in each case, the voltage response is consistent over the 2400 cycles. At a bending strain of 0.116%, the voltage response is consistently about +/−50 V. At a bending strain of 0.153%, the voltage response is consistently about +/−65 V. At a bending strain of 0.185%, the voltage response is consistently about +/−80 V, and at a bending strain of 0.214%, the voltage response is consistently about +/−95 V. Thus, the wearable device 512 can provide stable readings for various strains detected by the sensor 500.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred. In addition, as used herein, the article “a” is intended to include one or more than one component or element, and is not intended to be construed as meaning only one.

It will be apparent to those skilled in the art that various modifications and variations can be made without departing from the spirit or scope of the disclosed embodiments. Since modifications, combinations, sub-combinations, and variations of the disclosed embodiments incorporating the spirit and substance of the embodiments may occur to persons skilled in the art, the disclosed embodiments should be construed to include everything within the scope of the appended claims and their equivalents.

What is claimed is:

1. A sensor, comprising:
a ceramic substrate comprising a first major surface, a second major surface opposite to the first major surface, and a thickness measured from the first major surface to the second major surface, the thickness ranging from 10 μm to 200 μm;
a piezoelectric layer disposed on the first major surface of the ceramic substrate, the piezoelectric layer having a thickness of 10 μm or less; and
at least one electrical contact disposed on the piezoelectric layer or between the ceramic substrate and the piezoelectric layer or both on the piezoelectric layer and between the ceramic substrate and the piezoelectric layer.

2. The sensor of claim 1, wherein the piezoelectric layer comprises a transition metal dichalcogenide (TMD) material.

3. The sensor of claim 2, wherein the TMD material comprises at least one of molybdenum disulfide (MoS₂), tungsten disulfide (WS₂), tungsten diselenide (WSe₂), molybdenum ditelluride (MoTe₂), or tungsten ditelluride (WTe₂).

4. The sensor of claim 2, wherein the piezoelectric layer comprises a plurality of layers of the TMD material, the plurality of layers being up to eleven layers.

5. The sensor of any of claim 2, wherein the piezoelectric layer comprises a monolayer of the TMD material.

6. The sensor of claim 1, wherein the piezoelectric layer comprises at least one of lead zirconate titanate or lead magnesium niobate-lead titanate.

7. The sensor of claim 6, wherein the piezoelectric layer comprises a piezoelectric charge constant of at least 100 pC/N.

8. The sensor of any one of claim 1, wherein the ceramic substrate comprises at least one of silica, zirconia, or alumina.

9. The sensor of any one of claim 1, wherein the ceramic substrate comprises a grain size of 50 μm or less.

10. The sensor of claim 1, wherein the at least one electrical contact comprises interdigitated contacts.

11. The sensor of claim 1, further comprising a support extending from the second major surface of the ceramic substrate, the support forming a cavity disposed on the second major surface of the ceramic substrate.

12. A wearable device, comprising:
the sensor according to claim 1, wherein the ceramic substrate comprises zirconia.

13. The wearable device according to claim 12, wherein the zirconia is stabilized with 3 mol % of yttria.

14. The wearable device according to claim 12, wherein the piezoelectric layer comprises a piezoelectric charge constant of at least 100 pC/N.

15. The wearable device according to claim 12, wherein the sensor is laminated to at least one polymer layer.

16. A method of manufacturing a sensor, comprising:
sputtering a piezoelectric layer comprising a transition metal dichalcogenide (TMD) material onto a ceramic substrate using an RF magnetron, the ceramic substrate having a first major surface, a second major surface opposite to the first major surface, and a thickness between the first major surface and the second major surface, the thickness being from 10 μm to 200 μm, and the TMD material being disposed on the first major surface of the ceramic substrate;
heat treating the TMD material at a temperature in a range of 900° C. to 1100° C. for a time of from 15 minutes to 45 minutes.

17. The method of claim 16, wherein the treating further comprises flowing a gas containing a chalcogenide over the TMD material.

18. The method of claim 16, wherein the TMD material comprises molybdenum disulfide (MoS₂), tungsten disulfide (WS₂), tungsten diselenide (WSe₂), molybdenum ditelluride (MoTe₂), or tungsten ditelluride (WTe₂).

19. The method of claim 16, wherein sputtering produces a monolayer of the TMD material.

20. The method of claim 16, further comprising forming electrical contacts on the second major surface of the ceramic substrate before sputtering and/or forming the electrical contacts on the piezoelectric layer after the heat treating.

21. The method of claim 16, wherein the ceramic substrate comprises at least one of silica, zirconia, or alumina.

22. The method claim 16, further comprising laminating the sensor to at least one polymer layer.
